(19) United States
(12) Patent Application Publication
(10) Pub. No.: US 2016/0074453 A1
(43) Pub. Date: Mar. 17, 2016

(54) A METHOD AND COMPOSITION USING MYRRH, DILL AND PEEL OF POMEGRANATE AS A NATURAL REMEDY FOR CHRONIC WOUNDS

(71) Applicant: UMM AL-QURA UNIVERSITY, Makkah (SA)

(72) Inventor: Saeed Saeed ALGHAMDI, Makkah (SA)

(73) Assignee: UMM AL-QURA UNIVERSITY, Makkah (SA)

(21) Appl. No.: 14/387,954

(22) PCT Filed: Sep. 11, 2014
(86) PCT No.: PCT/IB2014/001802

§ 371 Date: Sep. 25, 2014

(51) Int. Cl. A61K 36/23; A61K 36/185; A61K 36/328

(52) U.S. Cl. CPC A61K 36/23; A61K 36/328; A61K 36/185

(57) ABSTRACT

A method of making a homogenized powder comprising anethum graveolens (dill), the resin/gum of the commiphora tree (myrrh), the peel of punica granatum (pomegranate) and/or an active ingredient derived therefrom, and topically administering it to an individual in amounts effective to inhibit and/or treat harmful effects of chronic wounds by helping tissue regeneration, preventing infections (being antiseptic), and having anti-biotic properties.

### BACKGROUND OF THE DISCLOSURE

1. Technical Field

The present disclosure relates to a composition comprising the herb anethum graveolens (dill), the resin/gum of the commiphora tree (myrrh), the peel of punica granatum (pomegranate) and/or an active ingredient derived therefrom and a method for administering the composition to a patient topically to inhibit and/or treat harmful effects of chronic wounds.

2. Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Dill, anethum graveolens is a species of anethum in the family apiaceae, and is native to southern Russia, western Africa and the Mediterranean region. It is widely known as an herb/spice/vegetable. Dill contains no cholesterol and is low in calories; however, it is a very rich herb in anti-oxidants/flavonoids, vitamins, minerals, essential volatile oils and dietary fibers. Minerals like copper, potassium, calcium, manganese, iron, and magnesium. Vitamins like folic acid, riboflavin, niacin, vitamin A, β-carotene, and vitamin-C. The herb contains health benefiting essential volatile oils that include d-carvone, dillapiole, DHC, eugenol, limonene, terpinene and myristicin.

Myrrh, the resin/gum of the commiphora tree is a species in the family burseraceae and is native to Yemen, Somalia, Eritrea and Eastern Ethiopia. The natural gum is ancient; it dates to thousands of years back. It has been widely used as a perfume, an incense and medicine. It is collected by purposefully and repeatedly wounding the commiphora tree through the bark into the sapwood; the tree then bleeds a waxy structured resin/gum that quickly hardens and becomes glossy. It is reddish in color and darkens as it ages.

Pomegranate, punica granatum is a fruit in the family of lythraceae and is commonly known to be native to Iran, but is currently being cultivated worldwide. Pomegranate is a fruit with a rich history. It has been mentioned in many ancient texts and meant different things in different cultures. It was considered a symbol of fertility, abundance, passion and happiness. The fruit is used for food, juice, flavor and color. It has great properties and is very nutritious both of which qualify it to be called a “super fruit”.

The fruit contains no cholesterol or saturated fats; it is moderate in calories and a rich source of soluble and insoluble dietary fibers. It is a good source of vitamins and anti-oxidants. The peel of pomegranate has double the anti-oxidants provided by the actual fruit. Antioxidants of the peel have higher activity level, and consist of the more powerful antioxidants such as flavonoids, phenolics, and proanthocyanidins. However, equal amounts of Vitamin C are in both in the fruit and the peel.

A composition containing dill, myrrh and pomegranate peel is described. The composition is shown to control, inhibit and/or heal chronic wounds, such as those caused by gangrene and diabetes. The composition in a conventional dosage form has characteristics different from modern medicines, such as consistency, reproducible quality, safety, physical, chemical and microbiological stability and bioavailability.

A chronic wound like diabetic foot/dry gangrene is a medical complication that is associated with diabetes. Diabetes is a disease where a person has high glucose concentration in the blood. A healthy human has a normal fasting glucose concentration that ranges from 70-100 mg/100 ml of blood and a mean normal blood glucose of about 100 mg/dL (5.5 mmol/L). A diabetic patient may experience poor blood circulation and nerve damage; loss of blood supply (lack of oxygen and nutrients to the cells) will lead to the death of body tissue or its prolonged healing and nerve damage will affect sensation and this generally affects fingers, toes and limbs. The term “diabetes” is used herein in the general sense of the disease diabetes mellitus including type 1 and type 2 diabetes and any other type, condition or complication related to the disease.

Wet gangrene is a condition that is a result of an infection, a burn or a trauma. An accident injury that crushes or squeezes a body part may result in rapid loss of blood supply associated with high chances of infections and other medical complications. It gets its name “wet” from the pus it forms. Such chronic wounds are considered more dangerous as they can quickly spread across the body and can become life-threatening if ignored.

The term “gangrene” is used herein in the general sense of the condition gangrene of tissue necrosis including dry, wet, gas, internal, necrotizing fasciitis and any other type, condition or complication related to this condition.

### BRIEF SUMMARY OF THE DISCLOSURE

The foregoing paragraphs have been provided by way of general introduction, and are not intended to limit the scope of the following claims. The described embodiments, together with further advantages, will be best understood by reference to the following detailed description and examples.

One embodiment relates to a method of making a homogenized powder comprising anethum graveolens (dill), the resin/gum of the commiphora tree (myrrh), the peel of punica granatum (pomegranate) and/or an active ingredient derived therefrom, and topically administering it to an individual in amounts effective to inhibit and/or treat harmful effects of chronic wounds.

In another embodiment, the method comprises dissolving or suspending the homogenized powder in a gel base composition to form a homogenized gel composition.

Another embodiment relates to a method that comprises topically administering the homogenized powder to a patient in an amount of at least 40 mg and up to 1 g per mm² of wound.

In another embodiment the homogenized powder prevents or treats the harmful effects of one or more medical conditions associated with diabetes including diabetic foot/dry gangrene.

In another embodiment the homogenized powder prevents or treats the harmful effects of one or more medical conditions associated with infections, burns and traumas to body parts including wet gangrene.

In another embodiment the homogenized powder is topically administered in a powder or gel base form.

In another embodiment the homogenized powder is in gel or ointment base formulation.

In another embodiment the homogenized powder is topically administered only after the wound is cleaned using alcohol.

In another embodiment the patient is not to be topically administered the homogenized powder for a period that is more than 30 consecutive days or less than 14 consecutive days.

### DETAILED DESCRIPTION OF THE EMBODIMENTS

The present disclosure relates to a method of making a homogenized powder comprising anethum graveolens (dill), the resin/gum of the commiphora tree (myrrh), the peel of punica granatum (pomegranate); and/or composition comprising anethum graveolens (dill), the resin/gum of the commiphora tree (myrrh), and the peel of punica granatum (pomegranate); and a method for treating chronic wounds comprising topically administering a homogenous powder comprising anethum graveolens (dill), the resin/gum of the commiphora tree (myrrh), and the peel of punica granatum (pomegranate) to an individual in amounts effective to inhibit and/or treat harmful effects of chronic wounds and/or to regenerate tissue, prevent infections, and provide anti-biotic properties.

The components anethum graveolens (dill (stems and leaves)), and the peel of punica granatum (pomegranate) were separately subjected to drying, grinding the dried material, and sieving the ground material to form two separate powders. The component resin/gum of the commiphora tree (myrrh) was subjected to grinding/pulverizing, and sieving the ground to a powder. All three resulting powders were mixed to form a homogenized powder.

In one embodiment of the present disclosure, the component anethum graveolens (dill (stems and leaves)), and the peel of punica granatum (pomegranate) may be freeze dried, sun dried, over dried or any other suitable method of drying but preferably the components are sun dried.

The homogenized powder includes anethum graveolens (dill), the resin/gum of the commiphora tree (myrrh), and the peel of punica granatum (pomegranate). Any suitable variety and/or amounts of these components may be of use in the present disclosure but preferably the amount being used is 50 grams of each of the components and, in any case, an amount effective for treating the harmful effects of chronic wounds. In another embodiment, the homogenate consists of only anethum graveolens (dill), the resin/gum of the commiphora tree (myrrh), and the peel of punica granatum (pomegranate) materials. In still another embodiment the homogenate consists essentially of the anethum graveolens (dill), the resin/gum of the commiphora tree (myrrh), and the peel of punica granatum (pomegranate) materials and excludes synthetic or non-naturally derived pharmaceutical agents.

In another embodiment, the ratio of the anethum graveolens (dill), the resin/gum of the commiphora tree (myrrh), and the peel of punica granatum (pomegranate) materials is 1:1:1 respectively. However, the weight ratio can range from 0.1-10:0.1-10:0.1-10, preferably 0.2-0.8:0.2-0.8:0.2-0.8; 0.3-0.7:0.3-0.7:0.3-0.7; or 0.4-0.6:0.4-0.6:0.4-0.6; respectively.

In one embodiment of this disclosure the anethum graveolens (dill) means the whole plant including roots, stems, leaves, flowers and seeds, but preferably the parts used are stems and leaves. The plant may be picked at any growth stage but preferably it is picked at a flowery stage close to its period of harvest, where it is considered to be moderately mature.

In another embodiment of this disclosure the resin/gum of the commiphora tree (myrrh) means the waxy structured resin/gum that is bled by the tree as a result of a wound through the bark, and that quickly hardens and becomes glossy. The resin/gum may be picked at any stage after two weeks of when the wound was first made but preferably it is picked after only two weeks and is allowed to further harden after being collected for a period of 8-14 weeks but preferably 12 weeks.

In another embodiment of this disclosure the peel of punica granatum (pomegranate) means the peel of the pomegranate plant only excluding any fruit, seeds or juice. The plant may be picked at any growth stage but preferably it is picked at a stage close to its period of harvest, where it is considered to be moderately mature.

In a preferable embodiment the homogenized powder is administered to a patient having a chronic wound twice a day only if the wound is cleaned with 50-80% alcohol or other anti-septic wound-cleansing agent such as boric acid, brilliant green, hydrogen peroxide, chlorhexidinegluconate, iodine, mercurochrome, or any other antiseptic agents but preferably 70% alcohol is used. The wound is preferably then covered with dry and clean gauze. The term “gauze” used herein is in the general sense of any thin natural or synthetic fabric made from any fiber in a plain or leno weave. It is made from a light material with an open weave to allow the wound to breathe and keep the material/fabric from sticking to the wound.

In another embodiment the gauze has a closed weave inhibiting or preventing the wound from breathing. For example, the wound is substantially isolated from contact with air.

In another embodiment the gauze used may or may not be in direct contact with the wound, but preferably it is not in direct contact with the wound.

In another embodiment the gauze can be made out of cotton and can be coated with materials like calamine and zinc oxide to help the healing process. The gauze may be of any size, shape or length but preferably a size, shape and length that are suitable for the size of the wound being treated.

The homogenized powder of anethum graveolens (dill), the resin/gum of the commiphora tree (myrrh), and the peel of punica granatum (pomegranate) materials can be prepared with acceptable properties for nutriceutical or pharmaceutical use. In one embodiment of the disclosure a homogenized powder that comprises processed anethum graveolens (dill (stems and leaves)), the resin/gum of the commiphora tree (myrrh), and the peel of punica granatum (pomegranate) materials, is suitable for nutriceutical or pharmaceutical use by topical administration.

Pharmaceutical agents suitable for incorporation into the homogenized powder include, but are not limited to, analgesics, decongestants, bronchodilators and other antiasthmatic agents, beta blockers, antihistamines, anesthetics, antifungals, antinauseants, antiemetics, antibacterial agents, antifungal agents, corticosteroids and anticonvulsants. The concentration of the active ingredient in the powder base is, of course, dependent on the identity of the active agent, and the condition patient being treated.

In one embodiment, the components of the homogenized powder are processed by grinding, crushing or grating or any other processing methods suitable for the present disclosure.

“Homogeneous” in this context means that the particles of anethum graveolens (dill (stems and leaves)), the resin/gum of the commiphora tree (myrrh), and the peel of punica granatum (pomegranate) materials present in the formulation are sufficiently fine and uniform that the powder feels smooth on the skin of the patient. In quantitative terms, “homogeneous” may mean that all or substantially all of the anethum graveolens (dill (stems and leaves)), the resin/gum of the commiphora tree (myrrh), and the peel of punica granatum (pomegranate) materials are present in the form of particles having a particle size of less than 500 μm, and more preferably less than 100 μm. For instance, more than 90% w/w of the anethum graveolens (dill (stems and leaves)), the resin/gum of the commiphora tree (myrrh), and the peel of punica granatum (pomegranate) materials are preferably present in the form of particles with a size less than 100 μm, or a size less than 80 μm, or a size less than 50 μm. It may well be that the majority of anethum graveolens (dill (stems and leaves)), the resin/gum of the commiphora tree (myrrh), and the peel of punica granatum (pomegranate) materials, e.g. more than 90% w/w or more than 95% w/w, is present in the suspension in the form of particles with a size in the range 1-50 μm.

The term “gel” is used herein in the general sense of a semi-solid, apparently homogeneous substance that may be elastic and jelly-like.

Another embodiment of this disclosure includes the composition in a gel base form including a range between 0.5 to 10.0% by weight CH₃(CH₂)₂₁(OCH₂CH₂)ₙ OH and from 90 to 99.5% of a glycol solvent, or from 2.5 to 10.0% by weight CH₃(CH₂)₂₁(OCH₂CH₂)ₙ OH and from 90 to 97.5% of a glycol solvent, wherein “n” is an integer from 2 to 30 and “x” is an integer from 15 to 20, the bases being substantially free of any added gelling agents. Preferred concentration ranges (by weight) for the components of the novel gels are from about 0.5 to about 2.5% of ethoxylated behenyl (C₂₂) alcohol and from about 97.5 to about 99.5% of a glycol solvent, or from about 2.5 to about 5% of a C₁₆-C₂₁ethoxylated alcohol and from about 95 to about 97.5% of a glycol solvent.

Lastly, the gel base further comprises the homogenized powder in an amount between about 5% and about 30%, and preferably between about 10% and about 20% by weight of the gel base.

Pharmaceutical agents suitable for incorporation into the gel base include, but are not limited to, analgesics, decongestants, bronchodilators and other antiasthmatic agents, beta blockers, antihistamines, anesthetics, antifungals, antinauseants, antiemetics, antibacterial agents, antifungal agents, corticosteroids and anticonvulsants. The concentration of the active ingredient in the gel base is, of course, dependent on the identity of the active agent, and the condition patient being treated.

The gel bases of the present disclosure may be prepared by any conventional method suitable for combining the ethoxylated alcohol surfactant component with the glycol solvent. But preferably, the glycol, for example propylene glycol or a polyethylene glycol having a molecular weight of about 200 to 800, is heated to about 80° C., and the surfactant is then stirred in with the mixture being immediately removed from heat. The mixture is allowed to cool to approximately 60°-65° C., at which point an active pharmaceutical ingredient may be stirred into the gel base until a homogeneous mixture, solution or suspension is achieved.

When active pharmaceutical agents (homogenized powder) are incorporated into the gel bases to form gel-like pharmaceutical compositions, the viscosities of said compositions range from about 2.5 to about 7.5 centipoises, with the polyethylene glycol-containing compositions generally exhibiting higher viscosities.

Embodiments of the stable topical pharmaceutical composition may include one or more pharmaceutically acceptable excipients. For example, the pharmaceutically acceptable excipients may include one or more of oils, lipids, stabilizers, emulsifiers, pH adjusting agents, emollients, humectants, preservatives, stabilizers, antioxidants, chelating agents, initiators, thickening agents, and the like.

The term “ointment” is used herein in the general sense of the stable mixture of ingredients of the final product.

Another embodiment of this disclosure includes the composition in an ointment base form including at least about 10% by weight of each of water, one or more paraffins, and a liquid polyol; less than about 10% by weight of each of beeswax, cetostearyl alcohol, a 4-hydroxy benzoic acid lower alkyl ester, a surface active agent, and a dispersing agent. In a preferred embodiment, the paraffin component includes both liquid paraffin and soft paraffin (white petrolatum), the liquid polyol component is glycerol, the beeswax component is white beeswax, the 4-hydroxy benzoic acid lower alkyl ester component is methyl parabens, the surface active agent is polyoxyethlene sorbitan monostearate, and the dispersing agent is glyceryl monostearate.

The paraffin component is present in a total amount of at least about 10%, and preferably at least about 12%, by weight of the ointment base. The paraffin ingredient preferably comprises liquid paraffin, present in an amount between about 3% and 8%, and most preferably between about 4% and 6% by weight of the ointment base. The paraffin ingredient preferably further comprises white petrolatum, also known as “soft paraffin”, in an amount preferably between 5% and 15%, and most preferably between 8% and 12% by weight of the ointment base.

The polyol component of the preferred ointment base comprises glycerin (also known as glycerol) as a water miscible cosolvent that can serve as a humectant in order to withdraw moisture from the skin. Glycerol is generally included at a concentration between about 20% and about 45%, and preferably between about 25% and about 35%, by weight of the ointment base.

The ointment base of the present invention further comprises beeswax, and preferably “white” beeswax, present in a concentration of between about 0.1% and 1.0%, preferably between about 0.3% and 0.5% by weight of the ointment base.

The ointment base further comprises cetostearyl alcohol in an amount between about 6% and about 10%, and preferably between about 7% and about 9% by weight of the ointment base. Cetostearyl alcohol is a mixture chiefly of hexadecyl and octadecanyl alcohols, and is commercially available in forms suitable for use in ointments.

The ointment base further comprises a 4-hydroxy benzoic acid lower alkyl ester (“parabens”) as a preservative. A preferred parabens is methyl paraben (i.e., p-hydroxybenzoic acid methyl ester), present in an amount between about 0.1% and about 0.5%, and preferably between about 0.3% and about 0.4% by weight of the ointment base.

The ointment base further comprises a surface active agent, preferably from the class known as polyoxyethylene sorbitan esters. Such surface active agents are preferably present at a concentration of between about 1% and about 10%, and more preferably between about 2% and about 5%, by weight, based on the weight of the ointment base. Those esters commonly known as “Polysorbates”, or in the alternative “Tweens”, are preferred. Particularly preferred is Polysorbate 60, a polyoxyethylene sorbitan ester having a fatty acid composition of about 50% stearic acid (polyoxyethylene sorbitan monostearate) and the balance primarily palmitic acid.

The ointment base of the present invention further comprises a dispersing agent, preferably in the form of glyceryl monostearate. Glyceryl monostearate is preferably present in an amount between about 0.5% and about 5%, and preferably between about 1% and about 3% by weight of the ointment base.

Lastly, the ointment base further comprises the homogenized powder in an amount between from 1 to 50% by weight based on the total weight of the ointment base, preferably from 5 to 40, 10 to 30, or 15-20% by weight. In another embodiment the ointment base further comprises the homogenized powder in an amount between from 0.1 to 10% by weight based on the total weight of the ointment, preferably from 0.5 to 5, 1.0 to 3.0, or about 2.0% by weight.

The ointment bases of the present disclosure may be prepared by any conventional method suitable but preferably following the steps below:

Step I—Preparation of Water Phase

Purified water, polysorbate, and glycerin were added with agitation to a standard laboratory mixing kettle. The contents were heated. Methylparaben was added, and the composition was mixed to dissolve this ingredient while maintaining temperature.

Step II—Preparation of Oil Phase

In a suitable vessel liquid paraffin, alcohol, white petrolatum, glycerol monostearate, and white beeswax were combined and were continuously mixed as the composition was heated.

Step III—Mixing of Phases

The mixture of Step II was transferred to the side feed container of the kettle, with the water phase maintained under 300 millibar vacuum. With mixing, and maintaining the temperature, the oil phase was drawn into the water phase. The resulting composition was mixed for some time with agitation and vacuum at 300 millibar. While mixing and under vacuum, the mixture was allowed to cool, then was allowed to continue cooling with agitation. The mixture was finally cooled to room temperature, where upon the vacuum was released, and the resulting ointment base was packaged in bulk.

In another embodiment, the ointment base may be prepared by dissolving the homogenate powder in olive oil in an amount of 350-600 ml, preferably between 400 and 550 ml; or 500 ml having a range of 3-12%, preferably 4-11%; or 10% of each of the anethum graveolens (dill), the resin/gum of the commiphora tree (myrrh), the peel of punica granatum (pomegranate).

Topical administrating methods of the gel and ointment base homogenized powder include squeezing the drug-containing gel directly from a tube or other container onto an affected tissue area and spreading a thin film of the gel and/or the ointment over that tissue area for treatment. Other suitable uses and routes of administration for the gel and/or ointment base homogenized powder will be apparent to those skilled in the medical and pharmaceutical arts.

Accordingly in a preferable embodiment, the patients are topically administered one of the homogenized powder, homogenized powder in the gel base form, or homogenized powder in the ointment base form in an amount of at least 40 mg and at most 1 g per cm² of the wound, depending on the patient's condition and the size of the wound. The topical administration to the wound is done twice a day, and only if the wound is cleaned with 50-80% alcohol or other anti-septic wound-cleansing agent such as boric acid, brilliant green, hydrogen peroxide, chlorhexidine gluconate, iodine, mercurochrome, or any other antiseptic agents but preferably 70% alcohol is used. The treatment period may be 10-30 consecutive days, but preferably the period is no longer than 30 consecutive days nor less than 14 consecutive days.

The following examples provide detailed illustrations of the compositions and methods of the present disclosure. These examples are not intended, however, to limit or restrict the scope of the invention in any way, and should not be construed as providing methods, conditions, ingredients or starting materials which must be utilized exclusively to practice the present disclosure.

### Examples

The homogenized powder was prepared and topically administered to 150 patients with chronic wounds aged between 34-71 years old. 72 patients were diagnosed with wet gangrene and 78 patients with dry gangrene/diabetic foot. Out of the 150 cases, 34 were acute wounds (trauma), while 116 were large wounds and below are the clinical data that reflects the effectiveness of the previously described homogenized powder.

| | | | | The site of | Current |
|---|---|---|---|---|---|
| | Gender | Age | Diagnosis | wound | status |
| 1 | Male | 55 | Diabetic foot | fingers | Cured |
| 2 | Male | 60 | Diabetic foot | fingers | Cured |
| 3 | Male | 39 | Diabetic foot | fingers | Cured |
| 4 | Male | 54 | Diabetic foot | fingers | Cured |
| 5 | Male | 57 | Diabetic foot | fingers | Cured |
| 6 | Male | 72 | Diabetic foot | fingers | Cured |
| 7 | Male | 54 | Diabetic foot | fingers | Cured |
| 8 | Male | 66 | Diabetic foot | fingers | Cured |
| 9 | Male | 67 | Diabetic foot | fingers | Cured |
| 10 | Male | 69 | Diabetic foot | fingers | Cured |
| 11 | Male | 71 | Diabetic foot | fingers | Cured |
| 12 | Male | 55 | Diabetic foot | fingers | Cured |
| 13 | Male | 45 | Diabetic foot | fingers | Cured |
| 14 | Male | 43 | Diabetic foot | fingers | Cured |
| 15 | Male | 57 | Diabetic foot | fingers | Cured |
| 16 | Male | 70 | Diabetic foot | fingers | Cured |
| 17 | Male | 39 | Diabetic foot | fingers | Cured |
| 18 | Male | 44 | Diabetic foot | fingers | Cured |
| 19 | Male | 73 | Diabetic foot | fingers | Cured |
| 20 | Male | 55 | Diabetic foot | fingers | Cured |
| 21 | Male | 64 | Diabetic foot | fingers | Cured |
| 22 | Male | 67 | Diabetic foot | fingers | Cured |
| 23 | Male | 57 | Diabetic foot | fingers | Cured |
| 24 | Male | 51 | Diabetic foot | fingers | Cured |
| 25 | Male | 48 | Diabetic foot | fingers | Cured |
| 26 | Male | 44 | Diabetic foot | fingers | Cured |
| 27 | Male | 46 | Diabetic foot | fingers | Cured |
| 28 | Male | 62 | Diabetic foot | fingers | Cured |
| 29 | Male | 58 | Diabetic foot | fingers | Cured |
| 30 | Male | 74 | Diabetic foot | fingers | Cured |
| 31 | Male | 66 | Diabetic foot | fingers | Cured |
| 32 | Male | 64 | Diabetic foot | fingers | Cured |
| 33 | Male | 68 | Diabetic foot | fingers | Cured |
| 34 | Male | 65 | Diabetic foot | fingers | Cured |
| 35 | Male | 64 | Diabetic foot | fingers | Cured |
| 36 | Male | 55 | Diabetic foot | fingers | Cured |
| 37 | Male | 67 | Diabetic foot | fingers | Cured |
| 38 | Male | 77 | Diabetic foot | fingers | Cured |
| 39 | Male | 73 | Diabetic foot | fingers | Cured |
| 40 | Male | 52 | Diabetic foot | fingers | Cured |
| 41 | Male | 63 | Diabetic foot | fingers | Cured |
| 42 | Male | 66 | Diabetic foot | fingers | Cured |
| 43 | Male | 67 | Diabetic foot | fingers | Cured |
| 44 | Male | 53 | Diabetic foot | fingers | Cured |
| 45 | Male | 56 | Diabetic foot | fingers | Cured |
| 46 | Male | 65 | Diabetic foot | fingers | Cured |
| 47 | Male | 67 | Diabetic foot | fingers | Cured |
| 48 | Male | 48 | Diabetic foot | fingers | Cured |
| 49 | Male | 47 | Diabetic foot | fingers | Cured |
| 50 | Male | 59 | Diabetic foot | fingers | Cured |
| 51 | Male | 53 | Diabetic foot | fingers | Cured |
| 52 | Male | 67 | Diabetic foot | fingers | Cured |
| 53 | Male | 55 | Diabetic foot | fingers | Cured |
| 54 | Male | 76 | Diabetic foot | fingers | Cured |
| 55 | Male | 71 | Diabetic foot | fingers | Cured |
| 56 | Male | 67 | Diabetic foot | fingers | Cured |
| 57 | Male | 69 | Diabetic foot | fingers | Cured |
| 58 | Male | 68 | Diabetic foot | fingers | Cured |
| 59 | Male | 63 | Diabetic foot | fingers | Cured |
| 60 | Male | 47 | Diabetic foot | fingers | Cured |
| 61 | Male | 49 | Diabetic foot | fingers | Cured |
| 62 | Male | 59 | Diabetic foot | fingers | Cured |
| 63 | Male | 58 | Diabetic foot | fingers | Cured |
| 64 | Male | 74 | Diabetic foot | fingers | Cured |
| 65 | Male | 67 | Diabetic foot | fingers | Cured |
| 66 | Male | 66 | Diabetic foot | fingers | Cured |
| 67 | Male | 67 | Diabetic foot | fingers | Cured |
| 68 | Male | 59 | Diabetic foot | fingers | Cured |
| 69 | Male | 55 | Diabetic foot | fingers | Cured |
| 70 | Male | 67 | Diabetic foot | fingers | Cured |
| 71 | Male | 57 | Diabetic foot | fingers | Cured |
| 72 | Male | 75 | Diabetic foot | fingers | Cured |
| 73 | Male | 46 | Diabetic foot | fingers | Cured |
| 74 | Male | 47 | Diabetic foot | fingers | Cured |
| 75 | Male | 78 | Diabetic foot | fingers | Cured |
| 76 | Male | 67 | Diabetic foot | fingers | Cured |
| 77 | Male | 46 | Diabetic foot | fingers | Cured |
| 78 | Male | 67 | Diabetic foot | fingers | Cured |
| 79 | Male | 77 | Diabetic foot | foot's bottom | Cured |
| 80 | Male | 77 | Diabetic foot | foot's bottom | Cured |
| 81 | Male | 78 | Diabetic foot | foot's bottom | Cured |
| 82 | Male | 63 | Diabetic foot | foot's bottom | Cured |
| 83 | Male | 57 | Diabetic foot | foot's bottom | Cured |
| 84 | Male | 70 | Diabetic foot | foot's bottom | Cured |
| 85 | Male | 64 | Diabetic foot | foot's bottom | Cured |
| 86 | Male | 66 | Diabetic foot | foot's bottom | Cured |
| 87 | Male | 60 | Diabetic foot | foot's bottom | Cured |
| 88 | Male | 58 | Diabetic foot | foot's bottom | Cured |
| 89 | Male | 63 | Diabetic foot | foot's bottom | Cured |
| 90 | Male | 65 | Diabetic foot | ankle | Cured |
| 91 | Male | 62 | Diabetic foot | ankle | Cured |
| 92 | Male | 69 | Diabetic foot | ankle | Cured |
| 93 | Male | 71 | Diabetic foot | ankle | Cured |
| 94 | Male | 76 | Diabetic foot | ankle | Cured |
| 95 | Male | 59 | Diabetic foot | ankle | Cured |
| 96 | Male | 60 | Diabetic foot | ankle | Cured |
| 97 | Male | 65 | Diabetic foot | ankle | Cured |
| 98 | Male | 57 | Diabetic foot | ankle | Cured |
| 99 | Male | 54 | Diabetic foot | ankle | Cured |
| 100 | Male | 58 | Diabetic foot | ankle | Cured |
| 101 | Male | 49 | Diabetic foot | ankle | Cured |
| 102 | Male | 61 | Diabetic foot | ankle | Cured |
| 103 | Male | 63 | Diabetic foot | ankle | Cured |
| 104 | Female | 50 | Diabetic foot | fingers | Cured |
| 105 | Female | 49 | Diabetic foot | fingers | Cured |
| 106 | Female | 43 | Diabetic foot | fingers | Cured |
| 107 | Female | 52 | Diabetic foot | fingers | Cured |
| 108 | Female | 54 | Diabetic foot | fingers | Cured |
| 109 | Female | 49 | Diabetic foot | fingers | Cured |
| 110 | Female | 50 | Diabetic foot | fingers | Cured |
| 111 | Female | 60 | Diabetic foot | fingers | Cured |
| 112 | Female | 55 | Diabetic foot | fingers | Cured |
| 113 | Female | 58 | Diabetic foot | fingers | Cured |
| 114 | Female | 53 | Diabetic foot | fingers | Cured |
| 115 | Female | 54 | Diabetic foot | fingers | Cured |
| 116 | Female | 57 | Diabetic foot | fingers | Cured |
| 117 | Female | 49 | Diabetic foot | fingers | Cured |
| 118 | Female | 52 | Diabetic foot | fingers | Cured |
| 119 | Female | 51 | Diabetic foot | fingers | Cured |
| 120 | Female | 59 | Diabetic foot | fingers | Cured |
| 121 | Female | 54 | Diabetic foot | fingers | Cured |
| 122 | Female | 55 | Diabetic foot | fingers | Cured |
| 123 | Female | 54 | Diabetic foot | fingers | Cured |
| 124 | Female | 48 | Diabetic foot | fingers | Cured |
| 125 | Female | 60 | Diabetic foot | fingers | Cured |
| 126 | Female | 57 | Diabetic foot | fingers | Cured |
| 127 | Female | 59 | Diabetic foot | fingers | Cured |
| 128 | Female | 49 | Diabetic foot | fingers | Cured |
| 129 | Female | 55 | Diabetic foot | fingers | Cured |
| 130 | Female | 58 | Diabetic foot | fingers | Cured |
| 131 | Female | 53 | Diabetic foot | fingers | Cured |
| 132 | Female | 50 | Diabetic foot | fingers | Cured |
| 133 | Female | 52 | Diabetic foot | foot's bottom | Cured |
| 134 | Female | 61 | Diabetic foot | foot's bottom | Cured |
| 135 | Female | 60 | Diabetic foot | foot's bottom | Cured |
| 136 | Female | 58 | Diabetic foot | foot's bottom | Cured |
| 137 | Female | 53 | Diabetic foot | foot's bottom | Cured |
| 138 | Female | 60 | Diabetic foot | foot's bottom | Cured |
| 139 | Female | 59 | Diabetic foot | foot's bottom | Cured |
| 140 | Female | 58 | Diabetic foot | foot's bottom | Cured |
| 141 | Female | 57 | Diabetic foot | foot's bottom | Cured |
| 142 | Female | 56 | Diabetic foot | foot's bottom | Cured |
| 143 | Female | 50 | Diabetic foot | foot's bottom | Cured |
| 144 | Female | 48 | Diabetic foot | ankle | Cured |
| 145 | Female | 55 | Diabetic foot | ankle | Cured |
| 146 | Female | 53 | Diabetic foot | ankle | Cured |
| 147 | Female | 50 | Diabetic foot | ankle | Cured |
| 148 | Female | 61 | Diabetic foot | ankle | Cured |
| 149 | Female | 58 | Diabetic foot | ankle | Cured |
| 150 | Female | 50 | Diabetic foot | ankle | Cured |

Data shown in the table above reflects 100% success. All 150 patients experienced significant tissue regeneration (80-100% regeneration) with the topical administration of the homogenized powder of the current disclosure. It was noted that patients with worse conditions took a longer treatment period, more towards the higher end of the range (30 consecutive days) but eventually had the same result, while in most cases the wounds were cured in 14 consecutive days.

Experiments were carried on rats induced with gangrene using arichiodonic acid. The homogenized powder was used to treat the rats and they were all cured. The table below shows the experimental data:

| | | | The site of | Current |
|---|---|---|---|---|
| | Gender | Diagnosis | wound | status |
| 1 | Male | Arichidonic acid | feet | Cured |
| | | induced gangrene | | |
| 2 | Male | Arichidonic acid | feet | Cured |
| | | induced gangrene | | |
| 3 | Male | Arichidonic acid | feet | Cured |
| | | induced gangrene | | |
| 4 | Male | Arichidonic acid | feet | Cured |
| | | induced gangrene | | |
| 5 | Male | Arichidonic acid | feet | Cured |
| | | induced gangrene | | |
| 6 | Male | Arichidonic acid | feet | Cured |
| | | induced gangrene | | |
| 7 | Male | Arichidonic acid | feet | Cured |
| | | induced gangrene | | |
| 8 | Male | Arichidonic acid | feet | Cured |
| | | induced gangrene | | |
| 9 | Male | Arichidonic acid | feet | Cured |
| | | induced gangrene | | |
| 10 | Male | Arichidonic acid | feet | Cured |
| | | induced gangrene | | |
| 11 | Female | Arichidonic acid | feet | Cured |
| | | induced gangrene | | |
| 12 | Female | Arichidonic acid | feet | Cured |
| | | induced gangrene | | |
| 13 | Female | Arichidonic acid | feet | Cured |
| | | induced gangrene | | |
| 14 | Female | Arichidonic acid | feet | Cured |
| | | induced gangrene | | |
| 15 | Female | Arichidonic acid | feet | Cured |
| | | induced gangrene | | |
| 16 | Female | Arichidonic acid | feet | Cured |
| | | induced gangrene | | |
| 17 | Female | Arichidonic acid | feet | Cured |
| | | induced gangrene | | |
| 18 | Female | Arichidonic acid | feet | Cured |
| | | induced gangrene | | |
| 19 | Female | Arichidonic acid | feet | Cured |
| | | induced gangrene | | |
| 20 | Female | Arichidonic acid | feet | Cured |
| | | induced gangrene | | |

Thus, the foregoing discussion discloses and describes merely exemplary embodiments of the present disclosure. As will be understood by those skilled in the art, the present disclosure may be embodied in other specific forms without departing from the spirit or essential characteristics thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A method for treating a wound that is a symptom of at least one of dry gangrene, wet gangrene and diabetes, comprising:
administering a composition comprising anethum graveolens (dill), the resin/gum of the commiphora tree (myrrh), the peel of punica granatum (pomegranate) to a wound of a human suffering from at least one of diabetes, dry gangrene, and wet gangrene, wherein the homogenized composition is topically administered in an amount effective to treat the wound,
wherein the homogenized composition is administered in an amount of at least 40 mg and at most 1 g of the homogenized composition/cm² the wound.

2. The method of claim 1 further comprising:
Different ratios of the anethum graveolens (dill), the resin/gum of the commiphora tree (myrrh), the peel of punica granatum (pomegranate).

3. The method of claim 1, wherein the composition is topically administered in an amount effective to enhance wound tissue regeneration from 80 to 100%, based on the tissue regeneration of the wound immediately before the topical administering and the tissue regeneration level of the wound 2 days after the topical administering.

4. The method of claim 1 wherein the homogenized composition is in a gel and/or ointment based form.

5. The method of claim 4 wherein the composition in the ointment form may be prepared by dissolving a predetermined amount of the homogenized composition in a corresponding predetermined amount of olive oil.

6. The method of claim 1 wherein the composition is topically administered to the wound in powder form.

7. The method of claim 1 wherein the composition is topically administered to the wound in gel form.

8. The method of claim 1 wherein the composition is topically administered to the wound in ointment form.

9. The method of claim 7 wherein the composition is topically administered to the patient in the gel base form and wherein the composition further comprises at least one pharmaceutically acceptable excipient selected from the group consisting of an oil, a lipid, a stabilizer, an emulsifier, a pH adjusting agent, an emollient, a humectant, a preservative, a stabilizer, an antioxidant, a chelating agent, an initiator, and a thickening agent.

10. The method of claim 8 wherein the composition is topically administered to the patient in the ointment base form and wherein the composition further comprises at least one pharmaceutically acceptable excipient consisting of an oil, a lipid, a stabilizer, an emulsifier, a pH adjusting agent, an emollient, a humectant, a preservative, a stabilizer, an antioxidant, a chelating agent, an initiator, and a thickening agent.

11. The method of claim 1 wherein the composition is topically administered to the wound only if the wound has been cleaned using alcohol prior to the administering.

12. The method of claim 11 wherein the homogenized composition is administered to the wound twice a day and is covered with clean gauze after every administration.

13. The method of claim 1 wherein the composition is topically administered to the wound for a minimum period of 14 consecutive days and a maximum period of 30 consecutive days.
